(19) United States
(12) Patent Application Publication
(10) Pub. No.: US 2011/0237810 A1
(43) Pub. Date: Sep. 29, 2011

(54) METHOD FOR PRODUCING TITANOSILICATE

(75) Inventors: Tomonori Kawabata, Osaka (JP); Makoto Yako, Wakayama (JP)

(73) Assignee: SUITOMO CHEMICAL COMPANY, LIMITED

(21) Appl. No.: 13/133,130

(22) PCT Filed: Dec. 4, 2009
(86) PCT No.: PCT/JP2009/070721

§ 371 Date: Jun. 6, 2011

(51) Int. Cl. C07D 301/12; C01B 39/00

(52) U.S. Cl. 549/531; 423/700; 423/706; 423/704

(57) ABSTRACT

The present invention relates to a method for producing a Ti-MWW precursor comprising: a first step of heating a mixture to prepare a solid, said mixture comprising a structure-directing agent, a compound containing a Group 13 element of the periodic table, a titanium-containing compound (1), a silicon-containing compound and water; and a second step of contacting the solid with a titanium-containing compound (2) and an inorganic acid.

### TECHNICAL FIELD

The present invention relates to a method for producing a titanosilicate and an oxidation reaction using the titanosilicate as a catalyst.

### BACKGROUND ART

With respect to an MWW type zeolite precursor which is a kind of titanosilicate, JP 2005-262164 A describes a method for producing a Ti-MWW precursor which comprises hydrothermally synthesizing a lamellar compound (which is also referred to as an as-synthesized sample) from a boron compound, tetrabutyl orthotitanate, fumed silica and piperidine, followed by contacting the lamellar compound with an aqueous solution of 2M nitric acid under a reflux condition, and a method for producing propylene oxide which comprises reacting propylene with hydrogen peroxide in the presence of the Ti-MWW precursor as a catalyst.

### SUMMARY OF INVENTION

The present invention provides a novel method for producing a Ti-MWW precursor and a method for producing an oxidized compound in which the Ti-MWW precursor is used.

Specifically, the present application relates to the following inventions:
[1] A method for producing a Ti-MWW precursor, comprising: a first step of heating a mixture to prepare a solid, said mixture comprising a structure-directing agent, a compound containing a Group 13 element of the periodic table, a titanium-containing compound (1), a silicon-containing compound and water; and a second step of contacting the solid with a titanium-containing compound (2) and an inorganic acid.
[2] The method according to [1], wherein the structure-directing agent is piperidine, hexamethyleneimine or a mixture thereof.
[3] The method according to [1], wherein the compound containing a Group 13 element of the periodic table is a boron compound.
[4] A catalyst for producing an oxidized compound, comprising the Ti-MWW precursor obtained by the method according to the above [1] or a silylated product thereof.
[5] A method for producing an oxidized compound, comprising a step of carrying out a reaction between an oxidant and an organic compound in the presence of a Ti-MWW precursor obtained by the method according to the above [1] or a silylated product thereof.
[6] A method for producing a titanosilicate having an MWW structure (which, hereinafter, may be referred to as a “Ti-MWW”), comprising a step of subjecting a Ti-MWW precursor obtained by the method according to the above [1] to dehydrative condensation.
[7] A catalyst for producing an oxidized compound, comprising a Ti-MWW obtained by the method according to the above [6].
[8] A method for producing an oxidized compound, comprising a step of carrying out a reaction between an oxidant and an organic compound in the presence of a Ti-MWW obtained by the method according to the above [6].
[9] The method according to [5] or [8], wherein the oxidant is oxygen or a peroxide.
[10] The method according to [9], wherein the peroxide is selected from the group consisting of hydrogen peroxide, tert-butyl hydroperoxide, tert-amyl hydroperoxide, cumene hydroperoxide, methyl cyclohexyl hydroperoxide, tetralin hydroperoxide, isobutylbenzene hydroperoxide, ethylnaphthalene hydroperoxide and peracetic acid.
[11] The method according to [5] or [8], wherein the reaction is epoxidation of an olefin compound or hydroxylation of benzene or a phenol compound.
[12] The method according to [11], wherein the oxidant is hydrogen peroxide and the reaction is epoxidation of an olefin compound.
[13] The method according to [12], wherein the oxidant is hydrogen peroxide synthesized in the same reaction system as that of the epoxidation of an olefin compound.
[14] The method according to [5] or [8], wherein the reaction is carried out in the presence of an organic solvent selected from the group consisting of alcohol, ketone, nitrile, ether, aliphatic hydrocarbon, aromatic hydrocarbon, halogenated hydrocarbon and ester.
[15] The method according to [14], wherein the organic solvent is acetonitrile or tert-butanol.

### EFFECT OF THE INVENTION

The method of the present invention can easily provides a titanosilicate in which the amount of Ti present in the zeolite extra-framework is small. The titanosilicate exhibits good catalytic activity and excellent selectivity in the oxidation reaction.

The titanosilicate obtained by the method of the present invention is converted into a zeolite having an MWW structure, so-called Ti-MWW, by heat-treating the titanosilicate, and the Ti-MWW also exhibits good catalytic activity and selectivity in the oxidation reaction.

### BRIEF DESCRIPTION OF DRAWINGS

FIG. 1 shows each UV absorption spectrum of a Ti-MWW precursor (1), Ti-MWW precursor (3) and comparative compound i.

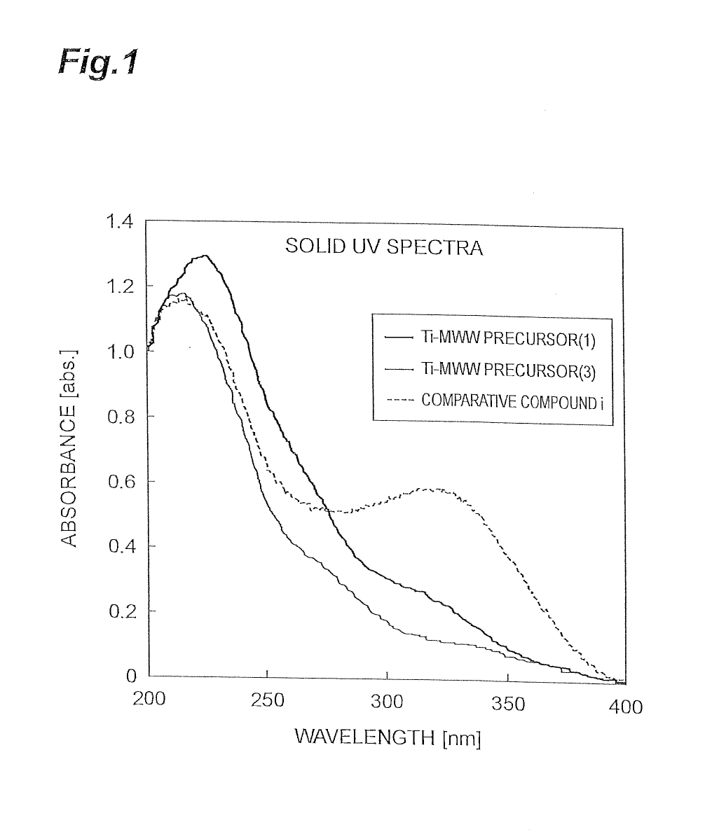

### DESCRIPTION OF EMBODIMENTS

The present invention will be described below in detail. Firstly, the first step in the method for producing a Ti-MWW precursor of the present invention will be described. The first step comprises heating a mixture. The mixture comprises a structure-directing agent, a compound containing a Group 13 element of the periodic table, a titanium-containing compound, a silicon-containing compound and water. Hereinafter, the “compound containing a Group 13 element of the periodic table” is sometimes referred to as a “Group 13 element-containing compound”.

Examples of the Group 13 element-containing compound include a boron-containing compound, an aluminum-containing compound and a gallium-containing compound, and preferred is a boron-containing compound. Examples of the boron-containing compound include boric acid; a borate salt; boron oxide; a boron halide; and a trialkyl boron compound having a C1-C4 alkyl group, and especially preferred is boric acid. The aluminum-containing compound includes sodium aluminate. The gallium-containing compound includes gallium oxide.

The amount of the Group 13 element-containing compound in the mixture of the first step is in the range of preferably 0.01 to 10 moles and more preferably 0.1 to 5 moles, based on one mole of silicon contained in the silicon-containing compound.

Examples of the silicon-containing compound include silicic acid, a silicate, silicon oxide, a silicon halide, a fumed silica compound, a tetraalkyl orthosilicate ester and a colloidal silica, and preferred is a fumed silica compound. The ratio of water to the silicon-containing compound in the mixture of the first step is in the range of preferably 5 to 200 moles and more preferably 10 to 50 moles, based on one mole of silicon.

Examples of the titanium-containing compound include a titanium alkoxide, a titanate, titanium oxide, a titanium halide, an inorganic salt of titanium and an organic salt of titanium, and preferred is a titanium alkoxide.

The titanium alkoxide includes a compound having a C1-C4 alkoxyl group, for example, titanium tetramethoxide, titanium tetraethoxide, titanium tetra-isopropoxide and titanium tetrabutoxide.

The organic salt of titanium includes titanium acetate. The inorganic salt of titanium includes titanium nitrate, titanium sulfate, titanium phosphate and titanium perchlorate. The titanium halide includes titanium tetrachloride. The titanium oxide includes titanium dioxide.

The amount of the titanium-containing compound (1) is in the range of usually 0.005 to 0.05 moles and preferably 0.01 to 0.05 moles, based on one mole of silicon in the silicon-containing compound.

In the present specification, a structure-directing agent means an organic compound contributing to the formation of a zeolite structure. The structure-directing agent can make polysilicate ions or polymetallo-silicate ions around it form a precursor of a zeolite structure (see Science and Engineering of Zeolite, pp. 33-34, Kodansha Scientific (2000)). The structure-directing agent is not particularly limited as long as it is a nitrogen-containing compound contributing to the formation of a zeolite having an MWW structure, and includes, for example, an organic amine such as piperidine and hexamethyleneimine; and a quaternary ammonium salt such as an N,N,N-trimethyl-1-adamantan-ammonium salt (such as N,N,N-trimethyl-1-adamantan-ammonium hydroxide, and N,N,N-trimethyl-1-adamantan-ammonium iodide) or an octyltrimethylammonium salt (such as octyltrimethylammonium hydroxide and octyltrimethylammonium bromide) which is described in Chemistry Letters, 916-917 (2007). These compounds may be used alone or may be used by mixing two or more kinds thereof at an arbitrary ratio. Preferred structure-directing agents are piperidine, hexamethyleneimine and a mixture thereof.

The amount of the structure-directing agent is in the range of preferably 0.1 to 5 moles and more preferably 0.5 to 3 moles, based on one mole of silicon in the silicon-containing compound.

The heating operation in the first step is preferably carried out by placing the mixture in a closed container such as autoclave under hydrothermal synthesis conditions where the mixture is heated and pressurized (see Chemistry Letters 774-775 (2000)). The temperature in the heating operation is in the range of preferably 110° C. to 200° C. and more preferably 120° C. to 180° C. The mixture after heating is usually separated into a solid component and a liquid component by filtering. The excessive raw material in the mixture after heating is separated by filtering. In addition, the solid component is washed with water or the like and dried by heating to obtain a lamellar compound. Here, the solid component is preferably washed until the pH of the washing solution reaches 10 to 11. The drying by heating is preferably carried out until the weight of the solid component is no longer reduced at a temperature of approximately 0° C. to 100° C.

Subsequently, the second step in the method for producing a Ti-MWW precursor of the present invention will be described. The second step comprises contacting the solid (hereinafter, referred to as a “solid (A)”) obtained in the first step with a titanium-containing compound (2) and an inorganic acid.

Examples of the inorganic acid in the second step include sulfuric acid, hydrochloric acid, nitric acid, perchloric acid, fluorosulfonic acid, and a mixture thereof. As the inorganic acid, nitric acid, perchloric acid, fluorosulfonic acid, and a mixture thereof are preferred.

The inorganic acid and solid (A) may be brought into contact together in a solution. Examples of the solvent in the solution include water, alcohol, ether, ester, ketone or a mixture thereof, and especially preferred is water.

When the organic acid is brought into contact with the solid (A), the concentration of the inorganic acid is not particularly limited and is in the range of usually 0.01 M to 20 M (M: mole/liter). A preferred concentration of the inorganic acid is 1 M to 5 M.

Examples of the titanium-containing compound (2) in the second step include a titanium alkoxide, titanium acetate, titanium nitrate, titanium sulfate, titanium phosphate, titanium perchlorate, a titanium halide such as titanium tetrachloride and titanium dioxide, and preferred is a titanium alkoxide. The titanium alkoxide includes a compound having a C1-C4 alkoxyl group, for example, titanium tetramethoxide, titanium tetraethoxide, titanium tetra-isopropoxide and titanium tetrabutoxide. The amount of the titanium-containing compound (2) is usually 0.001 to 10 parts by weight and preferably 0.01 to 2 parts by weight, based on 1 part by weight of the solid (A).

At contacting the solid (A) with the titanium-containing compound (2) and an inorganic acid, a mixture of the titanium-containing compound (2) and an inorganic acid is usually used. The temperature in the contact is preferably 20 to 150° C. and more preferably 50 to 104° C. The pressure in the contact is not particularly limited and is usually approximately 0 to 10 MPa in gauge pressure. The Ti-MWW precursor as obtained by the second step, which is a titanosilicate having a lamellar structure, is subjected to dehydrative condensation to form a Ti-MWW.

The structure of the Ti-MWW precursor can be confirmed by its X-ray diffraction pattern or ultraviolet-visible absorption spectrum described later. The X-ray diffraction pattern can be measured by using an X-ray diffraction apparatus capable of sending copper K-alpha radiation.

The Ti-MWW means a titanosilicate having an MWW structure (Chemistry Letters 774-775 (2000)). The MWW structure is a structure code of the International Zeolite Association (IZA). The titanosilicate has the structure in which some of Si atoms are isomorphously substituted with Ti atoms (see the description of the section of “Titanosilicate” in “Encyclopedia of Catalyst”, Asakura Publishing Co., Ltd., published on Nov. 1, 2000). The isomorphous substitution of Ti for Si can be easily confirmed, for example, by the presence of a peak of 210 to 230 nm in ultraviolet-visible absorption spectra. The ultraviolet-visible absorption spectra can be generally measured by a diffuse reflectance method with an ultraviolet-visible spectrophotometer equipped with a diffuse reflection apparatus.

The dehydrative condensation is usually carried out by heating the Ti-MWW precursor at a temperature of approximately 300° C. to 650° C.

The present application also includes a method for producing Ti-MWW, which comprises a step of subjecting a Ti-MWW precursor obtained by the method for producing a Ti-MWW precursor to dehydrative condensation.

The Ti-MWW precursor and the silylated product thereof as well as the Ti-MWW may be each used as a catalyst in a reaction such as oxidation reaction. A catalyst for producing an oxidized compound, which comprises the Ti-MWW precursor, the silylated product thereof or the TI-MWW, is also one of the present inventions. The catalyst of the present invention is useful for the oxidation reaction of an organic compound, especially for the epoxidation reaction of an olefin compound.

The silylated product of the Ti-MWW precursor may be obtained, for example, by silylating a Ti-MWW precursor with a silylating agent such as 1,1,1,3,3,3-hexamethyldisilazane.

The present application also includes a method for producing an oxidized compound which comprises a step of carrying out a reaction between an oxidant and an organic compound in the presence of a Ti-MWW precursor obtained by the production method for the Ti-MWW precursor or a silylated product thereof and a method for producing an oxidized compound which comprises a step of carrying out a reaction between an oxidant and an organic compound in the presence of a Ti-MWW obtained by the production method for the Ti-MWW.

Hereinafter, in the method for producing these oxidized compounds, a Ti-MWW precursor, a silylated product thereof and Ti-MWW are collectively referred to as “the present catalysts”.

In a method for producing an oxidized compound in the present invention, a reaction between an organic compound and an oxidant is carried out in the presence of a Ti-MWW precursor or a silylated product thereof or Ti-MWW.

In the present invention, an oxidant means a compound that provide oxygen atoms with an organic compound. The oxidant includes oxygen and a peroxide. Examples of the peroxide include hydrogen peroxide and an organic peroxide.

Examples of the organic peroxide include tert-butyl hydroperoxide, di-tert-butylperoxide, tert-amyl hydroperoxide, cumene hydroperoxide, methyl cyclohexyl hydroperoxide, tetralin hydroperoxide, isobutylbenzene hydroperoxide, ethylnaphthalene hydroperoxide and peracetic acid. As the oxidant, the peroxides exemplified above may be used by mixing two or more kinds thereof

As the peroxide, hydrogen peroxide is especially preferred. In the production method, hydrogen peroxide is used as an aqueous solution of hydrogen peroxide having a concentration of 0.0001% by weight or more to less than 100% by weight.

The hydrogen peroxide may be produced by a known method, and may be a commercially available one or may be one which is produced from oxygen and hydrogen in the presence of a noble metal in the same reaction system as that of the oxidation reaction. In the present invention, the amount of the oxidant may be arbitrarily selected depending on the kind of the organic compound or the reaction conditions or the like and is not particularly limited, but is preferably 0.01 parts by weight or more and more preferably 0.1 parts by weight or more, based on 100 parts by weight of the organic compound. As the amount of the oxidant, a preferred upper limit is 1000 parts by weight and a more preferred upper limit is 100 parts by weight, based on 100 parts by weight of the organic compound.

The organic compound in the above production method includes an aromatic compound such as benzene and a phenol compound; and an olefin compound.

Examples of the phenol compound include an unsubstituted or substituted phenol. Here, the substituted phenol means an alkyl phenol substituted with a C1-C6 linear or branched alkyl group or a C1-C6 cycloalkyl group. The linear or branched alkyl group includes a methyl group, an ethyl group, an isopropyl group, a butyl group and a hexyl group. The cycloalkyl group includes a cyclohexyl group.

The phenol compound includes, for example, 2-methylphenol, 3-methylphenol, 2,6-dimethylphenol, 2,3,5-trimethylphenol, 2-ethylphenol, 3-isopropylphenol, 2-butylphenol and 2-cyclohexylphenol, and especially preferred is phenol.

The olefin compound includes a compound in which a substituted or unsubstituted hydrocarbyl group or hydrogen is bonded to the carbon atoms forming the olefin double bond.

Examples of the substituent of the hydrocarbyl group include a hydroxyl group, a halogen atom, a carbonyl group, an alkoxycarbonyl group, a cyano group and a nitro group. Examples of the hydrocarbyl group include a saturated hydrocarbyl group, and examples of the saturated hydrocarbyl group include an alkyl group.

Examples of the olefin compound specifically include a C2-C10 alkene and a C4-C 10 cycloalkene.

Examples of the C2-C10 alkene include ethylene, propylene, butene, pentene, hexane, heptene, octene, nonene, decene, 2-butene, isobutene, 2-pentene, 3-pentene, 2-hexene, 3-hexene, 4-methyl-1-pentene, 2-heptene, 3 -heptene, 2-octene, 3-octene, 2-nonene, 3-nonene, 2-decene and 3-decene.

Examples of the C4-C10 cycloalkene include cyclobutene, cyclopentene, cyclohexene, cycloheptene, cyclooctene, cyclononene and cyclodecene.

In the present invention, as the organic compound, preferred is an olefin compound, more preferred is a C2-C10 alkene, still more preferred is a C2-C5 alkene, and especially preferred is propylene.

In the present invention, the amount of the organic compound may be arbitrarily selected depending on the kind of the organic compound or the reaction conditions or the like and is not particularly limited, but is preferably 0.01 parts by weight or more and more preferably 0.1 parts by weight or more, based on the total amount of 100 parts by weight of the solvent in the liquid phase. As the amount of the organic compound, a preferred upper limit is 1000 parts by weight and a more preferred upper limit is 100 parts by weight, based on the total amount of 100 parts by weight of the solvent in the liquid phase.

In the production method of the present invention, the amount of the present catalysts may be arbitrarily selected depending on the kind of the reaction, but the lower limit is generally 0.01 parts by weight, preferably 0.1 parts by weight and more preferably 0.5 parts by weight, and the upper limit is generally 20 parts by weight, preferably 10 parts by weight and more preferably 8 parts by weight, based on the total amount of 100 parts by weight of the solvent in the liquid phase.

The oxidation reaction in the present invention includes, for example, an epoxidation reaction of an olefin compound and a hydroxylation reaction of an aromatic compound such as benzene or a phenol compound.

The epoxidation reaction includes, for example, a reaction in which an olefin compound is converted into the corresponding epoxy compound. The hydroxylation reaction includes, for example, a reaction in which an aromatic compound is converted into phenol or a polyhydric phenol compound by the hydroxylation of the aromatic ring.

The production method of the present invention is suitable for epoxidation of a C2-C10 alkene, preferably a C2-C5 alkene and especially propylene using hydrogen peroxide as an oxidant.

In the production method of the present invention, an oxidized compound means an oxygen-containing compound obtained by the oxidation reaction. The oxidized compound includes an epoxy compound obtained by the epoxidation reaction and phenol or a polyhydric phenol compound obtained by the hydroxylation reaction.

In the production method for the oxidized compound, the conditions such as the reaction temperature and the reaction pressure may be arbitrarily set depending on the kind or amount or the like of the material to be used and is not limited.

As the reaction temperature, a preferred lower limit is 0° C., a more preferred lower limit is 40° C., a preferred upper limit is 200° C. and a more preferred upper limit is 150° C. As the reaction pressure, a preferred lower limit is 0.1 MPa, a more preferred lower limit is 1 MPa, a preferred upper limit is 20 MPa and a more preferred upper limit is 10 MPa. The recovery of the reaction product may be carried out by a well-known method such as separation by distillation.

In the production method of the present invention, the present catalysts are contacted with hydrogen peroxide in advance and then may be used for the reaction.

As the hydrogen peroxide in the contact, a hydrogen peroxide solution can be used. The concentration of the hydrogen peroxide solution is in the range of usually 0.0001% by weight to 50% by weight. The solution of hydrogen peroxide may be an aqueous solution or a solution of a solvent other than water. As the solvent other than water, a suitable solvent may be selected among the solvents for the oxidation reaction. The temperature of the contact is in the range of usually 0° C. to 100° C. and preferably 0° C. to 60° C.

In the production method of the present invention, when the oxidant is hydrogen peroxide, the hydrogen peroxide may be supplied by producing in the same reaction system as that of the oxidation reaction. When the hydrogen peroxide is produced in the same reaction system as that of the oxidation reaction, for example, the hydrogen peroxide may be produced from oxygen and water in the presence of a noble metal catalyst.

The noble metal catalyst includes a noble metal such as palladium, platinum, ruthenium, rhodium, iridium, osmium or gold, or an alloy or mixture thereof. Preferred noble metals include palladium, platinum and gold. A more preferred noble metal is palladium. As palladium, for example, a palladium colloid may be used (for example, see Examples 1 and the like in JP 2002-294301 A). As the noble metal catalyst, there may be used a noble metal compound which is converted into a noble metal by reducing in the oxidation reaction system.

When palladium is used as the noble metal catalyst, a metal other than palladium such as platinum, gold, rhodium, iridium and osmium may be further used by mixing with palladium. A preferred metal other than palladium includes gold and platinum.

An example of the noble metal compound includes a palladium compound. Examples of the palladium compound include tetravalent palladium compounds such as sodium hexachloro palladate (IV) tetrahydrate and potassium hexachloro palladate (IV); and divalent palladium compounds such as palladium (II) chloride, palladium (II) bromide, palladium (II) acetate, palladium (II) acetylacetate, dichlorobis(benzonitrile)palladium (II), dichlorobis(acetonitrile)palladium (II), dichloro(bis(diphenylphosphino)ethane)palladium (II), dichlorobis(triphenylphosphine)palladium (II), dichlorotetraamminepalladium (II), dibromotetraamminepalladium (II), dichloro(cycloocta-1,5-diene)palladium (II) and palladium trifluoroacetate (II).

A noble metal is generally used by being supported on a carrier. A noble metal may be used by being supported on the Ti-MWW precursor or may be used by being supported on an oxide of silica, alumina, titania, zirconia, niobia and the like, a hydrate of niobic acid, zirconium acid, tungstic acid, titanic acid and the like, carbon, and a mixture thereof. When a noble metal is supported on a carrier other than the Ti-MWW precursor, the carrier supported with the noble metal is mixed with the Ti-MWW precursor and the resulting mixture may be used as a catalyst. Among the carriers other than the Ti-MWW precursor, a preferred carrier is carbon. As a carbon carrier, activated carbon, carbon black, graphite, carbon nanotube and the like are known.

As a method of preparing a noble metal supported catalyst, for example, there is known a method of supporting a noble metal compound on a carrier and then reducing it. The supporting of the noble metal compound may be carried out by a conventionally well-known method such as an impregnation method.

As the reduction method, the reduction may be carried out by using a reducing agent such as hydrogen or by ammonia gas generated at the time of thermal cracking in an inert gas atmosphere. The reduction temperature varies depending on the kind of the noble metal compound, and when dichlorotetraamminepalladium (II) is used as a noble metal compound, the reduction temperature is in the range of preferably 100 to 500° C. and more preferably 200 to 350° C.

The noble metal supported catalyst contains a noble metal in the range of usually 0.01 to 20% by weight and preferably 0.1 to 5% by weight. The weight ratio of the noble metal to the Ti-MWW precursor (the weight of the noble metal/the weight of the Ti-MWW precursor) is preferably 0.01 to 100% by weight and more preferably 0.1 to 20% by weight.

Hereinafter, the production method of the present invention will be described in detail by taking as an example of a method of producing an epoxy compound by the oxidation (epoxidation) of an olefin compound.

In the production method, the reaction is generally carried out in a liquid phase containing a solvent. The solvent includes water, an organic solvent or a mixture of the both.

The organic solvent includes alcohols, ketones, nitriles, ethers, aliphatic hydrocarbons, aromatic hydrocarbons, halogenated hydrocarbons, esters and a mixture thereof. The aliphatic hydrocarbons include a C5-C 10 aliphatic hydrocarbon such as hexane and heptane. The aromatic hydrocarbons include a C6-C15 aromatic hydrocarbon such as benzene, toluene and xylene.

The alcohols include a C1-C6 monovalent alcohol and a C2-C8 glycol. As the alcohols, preferred is a C1-C8 aliphatic alcohol, more preferred is a C1-C4 monovalent alcohol such as methanol, ethanol, isopropanol and tert-butanol, and still more preferred is tert-butanol. As the nitriles, preferred are a C2-C4 alkyl nitrile such as acetonitrile, propionitrile, isobutyronitrile and butyronitrile and benzonitrile, and most preferred is acetonitrile.

As the organic solvent, preferred are alcohols and nitriles because of good catalytic activity and selectivity.

In the production method for an epoxy compound, if a buffering agent is allowed to exist in the reaction system, the reduction of the catalytic activity can be prevented, the catalytic activity can be further increased and the use efficiency of a raw material gas can be improved.

The buffering agent is generally allowed to exist in the reaction system by dissolving in a liquid phase. However, when hydrogen peroxide produced in the same reaction system is used as an oxidant, the buffering agent may be incorporated in a part of a noble metal complex in advance. For example, there is a method in which an ammine complex such as Pd tetramine chloride is supported on a carrier by an impregnation method and then the carrier is reduced to allow ammonia ions to remain, followed by generating a buffering agent during the epoxidation reaction. The amount added of the buffering agent is usually 0.001 mmol/kg to 100 mmol/kg per kg of solvent in the liquid phase.

An example of the buffering agent includes a buffering agent comprised of 1) an anion selected from the group consisting of a sulfate ion, a hydrogen sulfate ion, a carbonate ion, a hydrogen carbonate ion, a phosphate ion, a hydrogen phosphate ion, a dihydrogen phosphate ion, a hydrogen pyrophosphate ion, a pyrophosphate ion, a halogen ion, a nitrate ion, a hydroxide ion and a C1-C10 carboxylate ion, and 2) a cation selected from the group consisting of ammonium, C1-C20 alkyl ammonium, C7-C20 alkylaryl ammonium, an alkali metal and an alkali earth metal. The C1-C10 carboxylate ion includes an acetate ion, a formate ion, an acetate ion, a propionate ion, a butyrate ion, a valerate ion, a caproate ion, a caprylate ion, a caprate ion and a benzoate ion.

The alkyl ammonium includes tetramethylammonium, tetraethylammonium, tetra-n-propylammonium, tetra-n-butylammonium and cetyltrimethylammonium. Examples of the alkali metal and alkali earth metal cations include a lithium cation, a sodium cation, a potassium cation, a rubidium cation, a cesium cation, a magnesium cation, a calcium cation, a strontium cation and a barium cation.

Examples of a preferred buffering agent include an ammonium salt of an inorganic acid such as ammonium sulfate, ammonium hydrogen sulfate, ammonium carbonate, ammonium hydrogen carbonate, diammonium hydrogen phosphate, ammonium dihydrogen phosphate, ammonium phosphate, ammonium hydrogen pyrophosphate, ammonium pyrophosphate, ammonium chloride and ammonium nitrate, and an ammonium salt of a C1-C10 carboxylic acid such as ammonium acetate. A preferred ammonium salt includes ammonium dihydrogen phosphate.

In the method of producing an epoxy compound, when hydrogen peroxide, which is synthesized from oxygen and hydrogen in the same reaction system as that of the oxidation reaction, is used, the selectivity of the oxidized compound may be further increased by allowing a quinoid compound to exist in the reaction system.

Examples of the quinoid compound include a phenanthraquinone compound and a ρ-quinoid compound represented by the following formula (1):

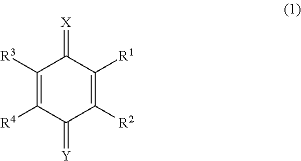

wherein, R¹, R², R³ and R⁴ represent a hydrogen atom, or R¹ and R², or R³ and R⁴ are independently bonded to each other at their terminal ends and represent a naphthalene ring which may be substituted together with the carbon atoms to which they are bonded; and X and Y are the same or different and represent an oxygen atom or an NH group.

Examples of the compound represented by the formula (1) include:
1) a quinone compound (1A) in which, in the formula (1), R¹, R², R³ and R⁴ are a hydrogen atom, and both X and Y are an oxygen atom;
2) a quinone-imine compound (1B) in which, in the formula (1), R¹, R², R³ and R⁴ are a hydrogen atom, X is an oxygen atom, and Y is an NH group; and
3) a quinone-diimine compound (1C) in which, in the formula (1), R¹, R², R³ and R⁴ are a hydrogen atom and both X and Y are an NH group.

The quinoid compound represented by the formula (1) includes an anthraquinone compound represented by the following formula (2):

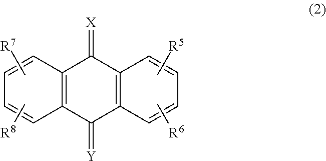

wherein X and Y are as defined in the formula (1); R⁵, R⁶, R⁷ and R⁸ are the same or different and represent a hydrogen atom, a hydroxyl group, or an alkyl group (for example, a C1-C5 alkyl group such as methyl, ethyl, propyl, butyl and pentyl).

In the formulas (1) and (2), X and Y preferably represent an oxygen atom.

The quinoid compound includes benzoquinone, naphthoquinone, anthraquinone, alkylanthraquinone compounds, polyhydroxyanthraquinone, ρ-quinoid compounds and o-quinoid compounds.

The alkylanthraquinone compound includes, for example, a 2-alkylanthraquinone compound such as 2-ethylanthraquinone, 2-tert-butylanthraquinone, 2-amylanthraquinone, 2-methylanthraquinone, 2-butylanthraquinone, 2-tert-amylanthraquinone, 2-isopropylanthraquinone, 2-s-butylanthraquinone or 2-s-amylanthraquinone; and a polyalkylanthraquinone compound such as 1,3-diethylanthraquinone, 2,3-dimethyl anthraquinone, 1 ,4-dimethyl anthraquinone and 2,7-dimethylanthraquinone. The polyhydroxyanthraquinone includes 2,6-dihydroxyanthraquinone. The ρ-quinoid compound includes naphthoquinone and 1,4-phenathraquionone. The o-quinoid compound includes 1,2-, 3,4- and 9,10-phenanthraquinone.

A preferred quinoid compound includes an anthraquinone or 2-alkylanthraquinone compound (in the formula (2), X and Y are a oxygen atom, R⁵ is an alkyl group substituted at 2 position, R⁶ represents a hydrogen atom, and R⁷ and R⁸ each represent a hydrogen atom).

The amount of the quinoid compound is usually 0.001 mmol/kg to 500 mmol/kg per kg of the solvent in the liquid phase. A preferred amount of the quinoid compound is 0.01 mmol/kg to 50 mmol/kg.

In the method of producing the oxidized compound, a salt comprised of ammonium, an alkylammonium or an alkylarylammonium can be simultaneously added into the reaction system.

The quinoid compound may be prepared by oxidizing a dihydro form of the quinoid compound using oxygen and the like in the reaction system. For example, a quinoid compound is generated to use by adding a compound obtained by hydrogenating a quinoid compound such as hydroquinone or 9,10-anthracenediol into a liquid phase and followed by oxidizing with oxygen in a reactor.

Examples of the dihydro form of the quinoid compound include compounds represented by the following formulas (3) and (4), which are dihydro forms of the compounds represented by the formulas (1) and (2):

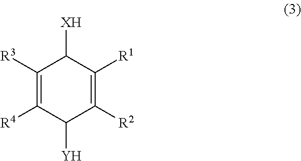

wherein, R¹, R², R³, R⁴, X and Y are as defined in the formula (1), and

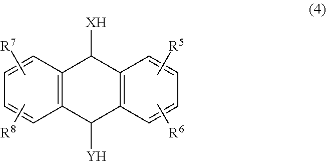

wherein, X, Y, R⁵, R⁶, R⁷ and R⁸ are as defined in the formula (2). In the formulas (3) and (4), X and Y preferably represent an oxygen atom.

A preferred dihydro form of the quinoid compound includes corresponding dihydro forms of the preferred quinoid compounds mentioned earlier.

The reaction method of the production method for the epoxy compound includes a fixed-bed flow reaction and a perfect mixing flow reaction of slurry.

In the case of a reaction in which an olefin compound is epoxidized by oxidation with a peroxide produced in advance, the reaction gas atmosphere is not limited.

In the case of producing a peroxide from oxygen and hydrogen in the presence of a noble metal in the same reaction system as that of the oxidation, the partial pressure ratio of oxygen and hydrogen fed to a reactor is in a range of usually 1:50 to 50:1. A preferable partial pressure ratio of oxygen and hydrogen is 1:2 to 10:1. When the partial pressure ratio of oxygen and hydrogen (oxygen/hydrogen) is too high, the production rate of an epoxy compound is decreased in some cases. When the partial pressure ratio of oxygen and hydrogen (oxygen/hydrogen) is too low, the selectivity of an epoxy compound decreases due to an increase in the amount of an alkane compound produced as a by-product in some cases.

In the present reaction, oxygen and hydrogen gases may have been diluted. A gas for dilution includes nitrogen, argon, carbon dioxide, methane, ethane and propane. Although the concentration of the gas for dilution is not limited, the reaction is carried out by diluting oxygen or hydrogen, where necessary.

The raw material of oxygen includes oxygen gas and air. As the oxygen gas, an inexpensive oxygen gas produced by a pressure swing method can be used, and a high purity oxygen gas produced by cryogenic separation can also be used as needed.

In the present epoxidation, the lower limit of the reaction temperature is usually 0° C., preferably 40° C. and more preferably 50° C., and the upper limit of the reaction temperature is usually 200° C., preferably 150° C. and more preferably 120° C. If the reaction temperature is too low, the reaction rate becomes slow, and if the reaction temperature is too high, the amount of by-products due to side reactions increases. The reaction pressure is usually 0.1 MPa to 20 MPa and preferably 1 MPa to 10 MPa in gauge pressure. The reaction product can be collected by a well-known method such as separation by distillation.

In the present epoxidation, although the amount of the present catalyst may be appropriately selected depending on the kind of the reaction, the lower limit is usually 0.01 parts by weight, preferably 0.1 parts by weight and more preferably 0.5 parts by weight and the upper limit is usually 20 parts by weight, preferably 10 parts by weight and more preferably 8 parts by weight, based on the total amount of 100 parts by weight of the solvent in the liquid phase.

In the present epoxidation, although the amount of the olefin compound may be arbitrarily selected depending on the kind of the olefin compound or the conditions of the reaction or the like, the lower limit is usually 0.01 parts by weight, preferably 0.1 parts by weight and more preferably 1 part by weight and the upper limit is usually 1000 parts by weight, preferably 100 parts by weight and more preferably 50 parts by weight, based on the total amount of 100 parts by weight of the solvent in the liquid phase.

In the present epoxidation, the amount of the oxidant may be arbitrarily selected depending on the kind of the olefin compound or the conditions of the reaction or the like, but is preferably 0.1 parts by weight or more and more preferably 1 part by weight or more, based on 100 parts by weight of the olefin compound. As the amount of the oxidant, a preferred upper limit is 100 parts by weight and a more preferred upper limit is 50 parts by weight, based on 100 parts by weight of the olefin compound.

### EXAMPLES

Hereinafter, the present invention will be described with Examples and is not limited to these Examples.

Analytical Apparatuses in Examples

### Elemental Analysis Method

The weight of titanium in a catalyst was determined by alkali fusion, nitric acid dissolution, and ICP emission spectrometry. Specifically, approximately 20 mg of a sample was weighed in a platinum crucible and sodium carbonate was placed on the sample, followed by performing fusion operation by a gas burner. After the fusion, the content in the platinum crucible was dissolved in purified water and nitric acid by heat, and then the solution as obtained was diluted to the constant volume with purified water. Thereafter, the quantitative determination of each element in the measurement solution was carried out using an ICP emission spectrometer (ICPS-8000, manufactured by Shimadzu Corporation).

Powder X-Ray Diffraction Method

The powder X-ray diffraction pattern of the sample was measured under the following conditions using the following apparatus.
Apparatus: RINT 2500V, manufactured by Rigaku Corporation
Radiation Source: Cu K-α radiation
Output: 40 kV-300 mA
Scanning range: 2θ=0.75 to 20°
Scanning Rate: 1°/min

Ultraviolet-Visible Absorption Spectrum (UV-Vis)

The sample was well pulverized by an agate mortar and then pelletized (7 mmφ) to prepare a sample for measurement. The ultraviolet-visible absorption spectra of the sample for measurement were measured under the following conditions using the following apparatus.
Apparatus: Diffuse Reflection Apparatus (Praying Mantis, manufactured by HARRICK Scientific Corp.)
Accessory: UV-VIS Spectrophotometer (V-7100, manufactured by JASCO)
Pressure: Atmospheric pressure
Measurement value: Reflectance
Data intake time: 0.1 sec
Band width: 2 nm
Measurement wavelength: 200 to 900 nm
Slit height: Half open
Data intake interval: 1 nm
Baseline correction (reference): BaSO₄ pellet (7 mmφ)

### Example 1

A gel was obtained by stirring 257 g of piperidine, 686 g of purified water, 6.4 g of tetra-n-butylorthotitanate (TBOT), 162 g of boric acid and 117 g of fumed silica (cab-o-sil M7D) at room temperature in an air atmosphere in an autoclave. The gel was aged for 1.5 hours and then the autoclave was closed. In addition, the gel was heated to 160° C. over 8 hours under stirring and maintained at 160° C. for 120 hours to obtain a suspension. The resulting suspension was filtered and then washed with water until the pH of the filtrate reached 10.2. The resulting solid content was dried at 50° C. until no reduction in the weight was observed to obtain 125 g of a solid 1a.

To 15 g of the solid 1a were added 750 mL of 2M nitric acid and 1.9 g of tetra-n-butylorthotitanate (TBOT) and the mixture was refluxed for 20 hours. Then, the resulting solid product was filtered and then washed with water until the pH of the filtrate became nearly neutral, followed by vacuum drying at 150° C. until no reduction in the weight was observed to obtain 12 g of a white powder. The X-ray diffraction pattern and ultraviolet visible absorption spectra of the white powder were measured, and the results showed that the white powder was confirmed to be a Ti-MWW precursor (hereinafter, referred to as a “Ti-MWW precursor (1)”). In addition, the titanium content by ICP emission analysis was 1.51% by weight.

### Example 2

9 g of Ti-MWW (hereinafter, referred to as “Ti-MWW (1)”) was obtained by heating 10 g of the Ti-MWW precursor (1) obtained in Example 1 at 530° C. for 6 hours. The fact that the resulting powder has an MWW structure was confirmed by measuring the X-ray diffraction pattern. The white powder was found to be a titanosilicate by the measurement results of the ultraviolet visible absorption spectra.

### Example 3

A gel was obtained by dissolving 899 g of piperidine, 2402 g of purified water, 112 g of tetra-n-butylorthotitanate (TBOT), 565 g of boric acid and 410 g of fumed silica (cab-o-sil M7D) under stirring at room temperature in an air atmosphere in an autoclave. The gel was aged for 1.5 hours and then the autoclave was closed. In addition, the gel was heated to 160° C. over 8 hours under stirring and maintained at 160° C. for 96 hours to obtain a suspension. The resulting suspension was filtered and then washed with water until the pH of the filtrate reached 10.7. The resulting solid content was dried at 50° C. until no reduction in the weight was observed to obtain 547 g of a solid 2a.

To 15 g of the solid 2a were added 750 mL of 2M nitric acid and 1.9 g of tetra-n-butylorthotitanate (TBOT) and the mixture was refluxed for 20 hours. Then, the resulting solid product was filtered and washed with water until the pH of the filtrate became nearly neutral, followed by vacuum drying at 150° C. until no reduction in the weight was observed to obtain 12 g of a white powder.

The X-ray diffraction pattern and ultraviolet visible absorption spectra of the white powder were measured, and the results showed that the white powder was confirmed to be a Ti-MWW precursor (hereinafter, referred to as a “Ti-MWW precursor (2)”). In addition, the titanium content by ICP emission analysis was 3.95% by weight.

### Example 4

A gel was obtained by stirring 257 g of piperidine, 686 g of purified water, 13.2 g of tetra-n-butylorthotitanate (TBOT), 162 g of boric acid and 117 g of fumed silica (cab-o-sil M7D) at room temperature in an air atmosphere in an autoclave. The gel was aged for 1.5 hours and then the autoclave was closed. In addition, the gel was heated to 160° C. over 8 hours under stirring and maintained at 160° C. for 120 hours to obtain a suspension. The resulting suspension was filtered and then washed with water until the pH of the filtrate reached 10.4. The resulting solid content was dried at 50° C. until no reduction in the weight was observed to obtain 145 g of a solid 3a.

To 75 g of the solid 3a were added 3750 mL of 2M nitric acid and 9.5 g of tetra-n-butylorthotitanate (TBOT) and the mixture was refluxed for 20 hours. Then, the resulting solid product was filtered and then washed with water until the pH of the filtrate became nearly neutral, followed by vacuum drying at 150° C. until no reduction in the weight was observed to obtain 49 g of a white powder. The X-ray diffraction pattern and ultraviolet visible absorption spectra of the white powder were measured, and the results showed that the white powder was confirmed to be a Ti-MWW precursor (hereinafter, referred to as a “Ti-MWW precursor (3)”).

### Example 5

A gel was obtained by stirring 257 g of piperidine, 686 g of purified water, 3.3 g of tetra-n-butylorthotitanate (TBOT), 162 g of boric acid and 117 g of fumed silica (cab-o-sil M7D) at room temperature in an air atmosphere in an autoclave. The gel was aged for 1.5 hours and then the autoclave was closed. In addition, the gel was heated to 160° C. over 8 hours under stirring and maintained at 160° C. for 120 hours to obtain a suspension. The resulting suspension was filtered and then washed with water until the pH of the filtrate reached 10.4. The resulting solid content was dried at 50° C. until no reduction in the weight was observed to obtain 137 g of a solid 4a.

To 75 g of the solid 4a were added 3750 mL of 2M nitric acid and 9.5 g of tetra-n-butylorthotitanate (TBOT) and the mixture was refluxed for 20 hours. Then, the resulting solid product was filtered and then washed with water until the pH of the filtrate became nearly neutral, followed by vacuum drying at 150° C. until no reduction in the weight was observed to obtain 61 g of a white powder. The X-ray diffraction pattern and ultraviolet visible absorption spectra of the white powder were measured, and the results showed that the white powder was confirmed to be a Ti-MWW precursor (hereinafter, referred to as a “Ti-MWW precursor (4)”). In addition, the titanium content by ICP emission analysis was 1.35% by weight.

### Comparative Example 1

A gel was obtained by stirring 899 g of piperidine, 2402 g of purified water, 22 g of tetra-n-butylorthotitanate (TBOT), 565 g of boric acid and 410 g of fumed silica (cab-o-sil M7D) at room temperature in an air atmosphere in an autoclave. The gel was aged for 1.5 hours and then the autoclave was closed. In addition, the gel was heated to 160° C. over 8 hours under stirring and maintained at 160° C. for 120 hours to obtain a suspension. The resulting suspension was filtered and then washed with water until the pH of the filtrate reached 10.4. The resulting solid content was dried at 50° C. until no reduction in the weight was observed to obtain 564 g of a solid 1b.

To 75 g of the solid 1b was added 3750 mL of 2M nitric acid and the mixture was refluxed for 20 hours. Then, the resulting solid product was filtered and then washed with water until the pH of the filtrate became nearly neutral, followed by vacuum drying at 150° C. until no reduction in the weight was observed to obtain 57 g of a white powder (compound a). The X-ray diffraction pattern and ultraviolet visible absorption spectra of the white powder were measured, and the results showed that the white powder was confirmed to be a Ti-MWW precursor.

18 g of a powder (compound b) was obtained by heating 20 g of the compound a at 530° C. for 6 hours. The fact that the compound b has an MWW structure was confirmed by measuring the X-ray diffraction pattern. The compound b was found to be a titanosilicate by the measurement results of the ultraviolet visible absorption spectra.

To 15 g of the compound b were added 750 mL of 2M nitric acid and 1.9 g of tetra-n-butylorthotitanate (TBOT), and the mixture was refluxed for 20 hours. Then, the resulting solid product was filtered and then washed with water until the pH of the filtrate became nearly neutral, followed by vacuum drying at 150° C. until no reduction in the weight was observed to obtain 16 g of a white powder (comparative compound i). The X-ray diffraction pattern and ultraviolet visible absorption spectra of the white powder were measured and the results showed that the white powder was confirmed to be a Ti-MWW. In addition, the titanium content by ICP emission analysis was 1.17% by weight.

Preparation of Pd/Activated Carbon (AC) Catalyst

A Pd/activated carbon (AC) catalyst was prepared by the following method.

Into a 1-L pear-shaped flask were added 3 g of activated carbon (produced by Wako Pure Chemical Industries Ltd.) which was preliminarily washed with 2 L of water and 300 mL of water, and the mixture was stirred at room temperature in air. To the resulting suspension was slowly dropwise added 100 mL of an aqueous solution containing 0.30 mmol of a Pd colloid (produced by JGC Catalysts and Chemicals Ltd.) at room temperature in air. After completion of the dropwise addition, the suspension was further stirred at room temperature in air for 8 hours. After completion of the stirring, water was removed with a rotary evaporator, and the residue was vacuum-dried at 80° C. for 6 hours and further heated at 300° C. for 6 hours in a nitrogen atmosphere to obtain a Pd/AC catalyst.

Hydrogen Peroxide Treatment of Titanosilicate

When used for purposes other than Example 6, all of the resulting titanosilicates obtained in Examples were subjected to hydrogen peroxide treatment according to the following method. The titanosilicates were treated with 100 g of a solution of water/acetonitrile=¼ (weight ratio) containing 0.1% by weight of hydrogen peroxide per 0.266 g of the titanosilicate at room temperature for one hour and then filtered, followed by washing with 500 mL of water.

### Example 6

An autoclave having a capacity of 0.3 L was used as a reactor. To the reactor was added the Ti-MWW precursor (1) (1.20 g) and were fed nitrogen at a rate of 500 mL/min, propylene at a rate of 2114 mmol/hr and a solution of 7% by weight of H₂O₂ (solvent: water/acetonitrile=20/80 (weight ratio)) at a rate of 559 mL/hr, and the reaction mixture was taken out through a filter from the reactor, thereby performing a continuous reaction under the conditions of a temperature of 60° C., a pressure of 3 MPa (gauge pressure) and a residence time of 10 minutes. The liquid and gas phases taken out 5.5 hours after the initiation of the reaction were analyzed by gas chromatography, and the results showed that the conversion rate of hydrogen peroxide was 98.2% and the production rate of propylene oxide was 661 mmol/hr.

### Example 7

An autoclave having a capacity of 0.5 L was used as a reactor. To the reactor were added the Ti-MWW precursor (1) (0.266 g) which was preliminarily subjected to hydrogen peroxide treatment and 0.03 g of the Pd/AC catalyst and were fed a raw material gas having a volume ratio of propylene/oxygen/hydrogen/nitrogen of 4/4/10/82 at a rate of 16 L/hr and 0.7 mmol/kg of anthraquinone and a solution of 1% by weight of propylene oxide (solvent: water/acetonitrile=20/80 (weight ratio)) at a rate of 108 mL/hr, and the reaction mixture was taken out through a filter from the reactor, thereby performing a continuous reaction under the conditions of a temperature of 60° C., a pressure of 0.8 MPa (gauge pressure) and a residence time of 90 minutes. The liquid and gas phases taken out 5 hours after the initiation of the reaction were analyzed by gas chromatography, and the results showed that the total production amount of propylene oxide and propylene glycol was 7.48 mmol/hr and the selectivity of propylene oxide, [the production amount of propylene oxide/(the production amount of propylene oxide and propylene glycol)]×100, was 89%.

### Example 8

Propylene oxide was produced in the same operation as in Example 7 except for using the Ti-MWW (1) instead of the Ti-MWW precursor (1). The liquid and gas phases taken out 6 hours after the initiation of the reaction were analyzed by gas chromatography, and the results showed that the total production amount of propylene oxide and propylene glycol was 7.31 mmol/hr and the selectivity of propylene oxide, [the production amount of propylene oxide/(the production amount of propylene oxide and propylene glycol)]×100, was 91%.

### Example 9

Propylene oxide was produced in the same operation as in

Example 7 except for using the Ti-MWW precursor (2) instead of the Ti-MWW precursor (1). The liquid and gas phases taken out 6 hours after the initiation of the reaction were analyzed by gas chromatography, and the results showed that the total production amount of propylene oxide and propylene glycol was 6.49 mmol/hr and the selectivity of propylene oxide, [the production amount of propylene oxide/(the production amount of propylene oxide and propylene glycol)]×100, was 92%.

### Example 10

Propylene oxide was produced in the same operation as in

Example 7 except for using the Ti-MWW precursor (3) instead of the Ti-MWW precursor (1). The liquid and gas phases taken out 6 hours after the initiation of the reaction were analyzed by gas chromatography, and the results showed that the total production amount of propylene oxide and propylene glycol was 6.16 mmol/hr and the selectivity of propylene oxide, [the production amount of propylene oxide/(the production amount of propylene oxide and propylene glycol)]×100, was 84%.

### Example 11

Propylene oxide was produced in the same operation as in Example 7 except for using the Ti-MWW precursor (4) instead of the Ti-MWW precursor (1). The liquid and gas phases taken out 6 hours after the initiation of the reaction were analyzed by gas chromatography, and the results showed that the total production amount of propylene oxide and propylene glycol was 4.64 mmol/hr and the selectivity of propylene oxide, [the production amount of propylene oxide/(the production amount of propylene oxide and propylene glycol)]×100, was 88%.

### Comparative Example 2

Propylene oxide was produced in the same operation as in Example 7 except for using the comparative compound i instead of the Ti-MWW precursor (1). The liquid and gas phases taken out 6 hours after the initiation of the reaction were analyzed by gas chromatography, and the results showed that the total production amount of propylene oxide and propylene glycol was 0.28 mmol/hr and the selectivity of propylene oxide, [the production amount of propylene oxide/(the production amount of propylene oxide and propylene glycol)]×100, was 82%.

### Example 12

By using a 30% H₂O₂ aqueous solution (produced by Wako Pure Chemical Industries Ltd.), acetonitrile and ion-exchanged water, a solution composed of 0.5% by weight of H₂O₂, 19.5% by weight of water and 80% by weight of acetonitrile was prepared. A 100-mL stainless autoclave was filled with 60 g of the solution prepared and 0.010 g of a Ti-MWW precursor (1) which was treated with hydrogen peroxide in advance. Then, the autoclave was transferred on an ice bath and then filled with 1.2 g of liquefied propylene. The reaction system was pressurized with argon to 2 MPa-G. The autoclave was placed in a hot water bath at 60° C. After one hour, the autoclave was taken out from the hot water bath, sampling was conducted and analysis was carried out using gas chromatography. The results showed that the production amount of propylene oxide was 4.66 mmol.

### Reference Example 1

Propylene oxide was produced in the same operation as in Example 12 except for using the compound a instead of the Ti-MWW precursor (1). As a result, the production amount of propylene oxide was 0.22 mmol.

On the other hand, the UV absorption spectrum was measured for each of the Ti-MWW precursor (1), Ti-MWW precursor (3) and comparative compound i. The absorbance (abs.) was obtained by applying the K-M transformation to the reflectance, and the results obtained by correcting the absorbance so that the absorbance at 200 nm is 1 are shown in Table 1.

It is clear that each spectrum of the Ti-MWW precursor (1) and Ti-MWW precursor (3) shows much absorbance in the vicinity of 210 nm to 230 nm corresponding to four-coordinate Ti species and less absorbance in 320 to 330 nm corresponding to extra-framework Ti species, compared to the comparative compound i obtained in Comparative Example 1.

### INDUSTRIAL APPLICABILITY

A titanosilicate obtained by the production method of the present invention exhibits good catalytic activity and excellent selectivity in the oxidation reaction, and may be used as a useful catalyst.

1. A method for producing a Ti-MWW precursor, comprising:
a first step of heating a mixture to prepare a solid, said mixture comprising a structure-directing agent, a compound containing a Group 13 element of the periodic table, a titanium-containing compound (1), a silicon-containing compound and water; and
a second step of contacting the solid with a titanium-containing compound (2) and an inorganic acid.

2. The method according to claim 1, wherein the structure-directing agent is piperidine, hexamethyleneimine or a mixture thereof

3. The method according to claim 1, wherein the compound containing a Group 13 element of the periodic table is a boron compound.

4. A catalyst for producing an oxidized compound, comprising a Ti-MWW precursor obtained by the method according to claim 1 or a silylated product thereof.

5. A method for producing an oxidized compound, comprising a step of carrying out a reaction between an oxidant and an organic compound in the presence of a Ti-MWW precursor obtained by the method according to claim 1 or a silylated product thereof.

6. A method for producing a titanosilicate having an MWW structure, comprising a step of subjecting the Ti-MWW precursor obtained by the method according to claim 1 to dehydrative condensation.

7. A catalyst for producing an oxidized compound, comprising a titanosilicate obtained by the method according to claim 6.

8. A method for producing an oxidized compound, comprising a step of carrying out a reaction between an oxidant and an organic compound in the presence of a titanosilicate obtained by the method according to claim 6.

9. The method according to claim 5, wherein the oxidant is oxygen or a peroxide.

10. The method according to claim 9, wherein the peroxide is selected from the group consisting of hydrogen peroxide, tert-butyl hydroperoxide, tert-amyl hydroperoxide, cumene hydroperoxide, methyl cyclohexyl hydroperoxide, tetralin hydroperoxide, isobutylbenzene hydroperoxide, ethylnaphthalene hydroperoxide and peracetic acid.

11. The method according to claim 5, wherein the reaction is epoxidation of an olefin compound or hydroxylation of benzene or a phenol compound.

12. The method according to claim 11, wherein the oxidant is hydrogen peroxide and the reaction is epoxidation of an olefin compound.

13. The method according to claim 12, wherein the oxidant is hydrogen peroxide synthesized in the same reaction system as that of the epoxidation of an olefin compound.

14. The method according to claim 5, wherein the reaction is carried out in the presence of an organic solvent selected from the group consisting of alcohol, ketone, nitrile, ether, aliphatic hydrocarbon, aromatic hydrocarbon, halogenated hydrocarbon and ester.

15. The method according to claim 14, wherein the organic solvent is acetonitrile or tert-butanol.

16. The method according to claim 8, wherein the oxidant is oxygen or a peroxide.
